(19) United States
(12) Patent Application Publication
(10) Pub. No.: US 2016/0081589 A1
(43) Pub. Date: Mar. 24, 2016

(54) Device for Analyzing Exhaled Air, and Use of the Device

(71) Applicant: Robert Bosch GmbH, Stuttgart (DE)

(72) Inventors: Daniel Reisinger, Rottenburg-Wendelsheim (DE); Peter Zeyher, Darmstadt (DE); Frank Barth, Tamm (DE); Robert Giezendanner-Thoben, Gerlingen (DE); Volker Wingsch, Reutlingen (DE)

(21) Appl. No.: 14/861,553

(22) Filed: Sep. 22, 2015

(30) Foreign Application Priority Data
Sep. 23, 2014 (DE) 10 2014 219 161.0

(51) Int. Cl. A61B 5/097; A61B 5/091; A61B 5/087; A61B 5/08

(52) U.S. Cl. CPC A61B 5/097; A61B 5/082; A61B 5/091; A61B 5/087

(57) ABSTRACT

A device for analyzing exhaled air comprises a gas sensor unit. The device is configured for measuring nitrogen oxides in the exhaled air. The device includes a main gas path in order to guide air in the device. At least one measuring gas path and at least one flushing gas path branch off from the main gas path.

This application claims priority under 35 U.S.C. §119 to patent application no. DE 10 2014 219 161.0, filed on Sep. 23, 2014 in Germany, the disclosure of which is incorporated herein by reference in its entirety.

The disclosure relates to a device for analyzing exhaled air and in particular for measuring nitrogen oxides in the exhaled air, wherein the device comprises a gas sensor unit with at least one gas sensor. The disclosure further relates to a use of the device.

### BACKGROUND

Various appliances are already used for respiratory gas analysis for medical purposes, in particular for diagnosis, and also for various lifestyle applications. With regard to asthmatic diseases, an important role is played by endogenous nitrogen monoxide in the air exhaled by a person. Nitrogen monoxide is an indicator of inflammatory reactions in the airways and, in particular, in the lungs. It is already known to monitor the nitrogen monoxide content in the exhaled air in order to achieve improved management of asthma patients. For this purpose, various respiratory air analysis appliances are known. For example, the US patent application US 2010/0192669 A1 describes a respiratory air analysis appliance with a photoacoustic sample detector with which nitrogen monoxide can be determined in the exhaled air. The German utility model DE 20 2009 018 824 U1 discloses a sampling system for an appliance for respiratory gas analysis, in which the flow of the respiratory air of the patient through the measuring appliance is controlled such that the clinically important fraction of the tidal volume can be conveyed through a measuring sensor. Here, the analysis appliance has a conduit system for the respiratory gas with a branched arrangement, wherein a first conduit branch leads to a measuring chamber with a gas sensor and a second conduit branch leads to a further outlet opening. To flush the measuring chamber, ambient air can be conveyed through a filter and a fan into the measuring chamber. The published document WO 02/088691 A2 of an international patent application describes a device for measuring nitrogen monoxide in exhaled air, wherein a gas sensor is used that is operated according to the principle of work function measurement. Here, an oxidation catalyst is provided as a converter, which oxidizes nitrogen monoxide to nitrogen dioxide. From the measured nitrogen dioxide concentration, it is possible to draw conclusions regarding the nitrogen monoxide concentration in the exhaled air. Field effect transistors are used here as gas sensors.

### SUMMARY

The disclosure makes available a device for analyzing exhaled air and in particular for measuring nitrogen oxides in the exhaled air, i.e. a respiratory gas analysis appliance, with which the nitrogen monoxide content in the air exhaled by a person, or by an animal, can be determined in a particularly advantageous manner. The device comprises a gas sensor unit, particularly in the form of a measuring chamber, which preferably comprises at least one gas sensor for measuring nitrogen oxides. According to the disclosure, a main gas path is provided for guiding air, in particular exhaled air, through the device, from which main gas path at least one measuring gas path and at least one flushing gas path branch off. Both the measuring gas path and also the flushing gas path open into the measuring chamber or into the gas sensor unit. It is expedient that only a fraction, for example 10%, of the exhaled air guided in the main gas path is fed into the measuring gas path or into the flushing gas path. The rest of the air stream leaves the device via an outlet opening of the main gas path. Thus, a partial stream of the exhaled air guided in the device is conveyed into the measuring chamber or into the gas sensor unit. The partial stream that is conveyed through the measuring gas path into the measuring chamber is used to measure the nitrogen oxides. The partial stream that is conveyed through the flushing gas path into the measuring chamber serves for flushing and/or optionally for calibration and/or for zero line adjustment of the gas sensor unit or of the gas sensor.

In a particularly preferred embodiment of the device, at least one filter is provided in the flushing gas path in order to generate air, substantially free of harmful substances, in the flushing gas path. The filter (zero air filter) can be an activated carbon filter, for example. The air free of harmful substances is characterized in particular in that it contains substantially no nitrogen oxides, in particular no nitrogen monoxide or nitrogen dioxide. Furthermore, the air free of harmful substances is preferably free of alcoholic components and/or of carbon monoxide. By filtering the air, it is thus possible to generate an air fraction which is substantially free of harmful substances and which is suitable as flushing air. As an alternative to using exhaled air for generating the flushing air, ambient air can also be sucked through the mouthpiece of the device with the aid of a pump. By flushing with the air substantially free of harmful substances, the exhaled air that remains in the measuring chamber can be flushed out after the measurement procedure. In addition, the gas sensor can be calibrated and/or the zero line of the gas sensor can be readjusted. Particularly preferably, at least one pump is provided for removing gas, in particular respiratory air, from the main gas path into the measuring gas path and into the flushing gas path. In principle, two configurations are particularly advantageous here. In a first configuration, a common pump can be provided for the measuring gas path and for the flushing gas path. In a second configuration, at least one pump is provided respectively in the measuring gas path and in the flushing gas path. By means of the pump or pumps, exact control of the sampling in respect of the time of the measurement and the volume of the sample is possible, as a result of which the measurement can be simplified and made precise. The active pumping of the air stream moreover affords the additional advantage that, as a result of the associated pressure increase in the system, the cross section of the conduits and valves used can be made smaller by comparison with passive systems.

In the embodiment with one pump, switching is expediently provided between the measuring gas path and the flushing gas path, for which purpose a switchover valve is preferably arranged in the conduit system. This can be, for example, a conventional electromechanical switchover valve. The common pump can in principle be arranged before or after the gas sensor unit, i.e. upstream or downstream from the gas sensor unit. The arrangement of the pump downstream from the gas sensor unit has the particular advantage of avoiding soiling of the gas sensor unit or of the measuring chamber, which soiling can be caused by the pump. In addition, with a pump arranged downstream from the gas sensor unit, an underpressure can be generated in the measuring chamber. In this way, flushing and regeneration of the sensor in the gas sensor unit can be accelerated, such that the system is ready again more quickly for the next measurement.

In the other embodiment of the device with at least one pump in the measuring gas path and at least one pump in the flushing gas path, the flow of gas through the measuring gas path and the flow of gas through the flushing gas path can be controlled in a particularly effective manner. In this embodiment, it is possible to dispense completely with a switchover valve. By using two optimal micropumps instead of the valve required in the other variant, the material costs can be reduced by more than 30%. The required installation space is also reduced in this variant.

The one or more pumps in the system can be, for example, diaphragm pumps and/or so-called microblowers. Microblowers are miniaturized air pumps that work with piezoceramic elements. These pumps are very suitable for small volumetric flows and, in addition, can be very precisely controlled. In addition, the design can be kept very small, such that these pumps are particularly suitable for the production of handheld appliances.

At least one nonreturn valve is expediently assigned to the pump or to each of the pumps. Depending on the type of pump, the nonreturn valve is arranged upstream or downstream from the pump. In the case where the pump is a microblower, the nonreturn valve, which is arranged upstream or downstream from the microblower, can advantageously be configured such that the nonreturn valve opens only when there is a pressure increase caused by the running pump, e.g. at an opening pressure of 0.3 kPa. This ensures that a stream of gas is conveyed into the measuring chamber or into the sensor unit only when the pump is running.

For reliable measurement results and also for user friendliness, it is important that the system has a good design in respect of the pressure conditions and the flow rates. In particular, the user or the patient should be able to breathe as comfortably as possible when the exhaled air is being blown into the appliance. If the air resistance is too high or if the exhalation is ended abruptly by a valve closure, this may be unpleasant for the patient during use and may in some circumstances also distort the measurement results. To ensure that the exhalation is not ended abruptly, the main gas path of the device according to the disclosure is equipped with a dedicated outlet opening, such that a certain flow of the exhaled air through the appliance is ensured at all times. For optimal operation of the device according to the disclosure, it is particularly suitable if an air stream in the main gas path measures 50 ml/s at a counterpressure for the patient of 0.5 to 2 kPa, as is recommended, for example, in the guideline ATS/ERS Recommendations for Standardized Procedures for the Online and Offline Measurement of Exhaled Lower Respiratory Nitric Oxide and Nasal Nitric Oxide (Am J Respir Crit Care Med Vol 171, pp. 912-930, 2005). Compared to passive systems, which operate without pumps, the device according to the disclosure, which uses one or more pumps, therefore has the advantage that suitable pressure conditions and flow rates can be very precisely adjusted.

Since, according to the disclosure, only a secondary stream or partial stream, for example 10%, of the exhaled air is conveyed into or through the measuring chamber or the gas sensor unit, the device according to the disclosure overall has the advantage that the system can be made relatively small and can therefore generally be made less expensive. For example, the switchover valve, which is necessary in the above-described variant with a common pump for the measuring gas path and the flushing gas path, can be made much smaller and therefore made less expensive than in other solutions. Other components, for example conduits, the pump(s), the measuring chamber, and the components contained therein, can also be made smaller than in other systems.

The device according to the disclosure expediently has a mouthpiece through which the air exhaled by the user is blown into the device. It can in particular be an exchangeable mouthpiece which, for example, is exchanged or renewed after a certain number of uses or after a defined time. Particularly preferably, the mouthpiece is equipped with one or more dehumidifiers which, for example, contain silica gel or water-binding salts. Removal of moisture from the exhaled air is particularly advantageous since condensation of moisture in the measuring chamber can distort the measurement result. In connection with the disclosure, removal of moisture from the exhaled air in the area of the mouthpiece also has the particular advantage that, in this way, moisture is also removed from the flushing air, which enters the device through this mouthpiece. Previous devices generally obtain the flushing air directly from the ambient air, such that the flushing air is generally not dehumidified or has to be dehumidified separately. By sufficient removal of moisture from the flushing air in the mouthpiece, it is possible to avoid a situation where condensation of moisture from the flushing air in the measuring chamber negatively influences the accuracy of the nitrogen oxide measurement.

Moreover, the mouthpiece can contain one or more microbe filters. This is advantageous, particularly in view of hygiene requirements placed on the device, since contamination and microbial colonization of the device are in this way avoided during prolonged use.

In a particularly preferred embodiment of the device according to the disclosure, the user is able to breathe in ambient air, preferably filtered ambient air, through the mouthpiece. For this purpose, a delivery path for ambient air opens into the mouthpiece. Particularly advantageously, at least one filter (zero air filter) is provided in this path for the purpose of generating air that is substantially free of harmful substances, such that the user can breathe in air free of harmful substances before he blows the exhaled air to be measured into the appliance. In this way, it is possible to rule out an offset of the measurement results by harmful substances from the ambient air. The filter can, for example, be an activated carbon filter which, in particular, filters out nitrogen oxides and/or alcoholic components and/or carbon monoxide from the ambient air. In this delivery path for ambient air, a nonreturn valve can optionally be provided in order to protect the filter from contamination during storage.

The branching of the measuring gas path and of the flushing gas path from the main gas path permits the use of various gas sensor units which are either based on a measurement in a measuring chamber through which there is a continuous flow or are based on a measurement using a closed-off sample volume. For the gas sensor unit, it is therefore possible in principle to use various gas sensors that are based on different principles and that are designed to detect and measure different components in the exhaled air.

Particularly advantageously, the device according to the disclosure is designed for measuring nitrogen oxides in exhaled air. For this purpose, a nitrogen oxide gas sensor can be used which, for example, uses absorption spectroscopy to directly measure nitrogen monoxide in the exhaled air. It is particularly preferable to use a gas sensor unit coupled to a converter, which at least partially converts the nitrogen monoxide from the exhaled air to nitrogen dioxide. In this embodiment, one or more gas sensors suitable for the measurement of nitrogen dioxide are then provided in the gas sensor unit. For the conversion of nitrogen monoxide to nitrogen dioxide, suitable oxidizing agents can be provided in a manner known per se in the converter, or the conversion to nitrogen dioxide takes place by means of a suitable catalyzer.

The gas sensor can, for example, operate according to the principle of work function measurement, wherein a field effect transistor is preferably used. A suitable gas sensor unit is found, for example, in the aforementioned international patent application WO 2002/088691 A2. A sensor of this type has the particular advantage that the sensor can have a very small and energy-efficient design. Moreover, the sensor does not suffer wear, and it is therefore particularly suitable for the device according to the disclosure.

The device according to the disclosure expediently has a suitable sensor system for controlling the flow of gas and the volumetric flow rates in the device. It is possible in particular to provide one or more sensors for measuring the quantity of air and/or the pressure and/or the flow of gas. In this way, an optimal air fraction or air quantity of the exhaled air blown into the appliance can be used for the measuring and for the flushing. A suitable sensor system thus permits optimal control of the sampling for controlled measurements. The sensors can be conventional flow sensors and/or pressure sensors. For example, a differential pressure measurement can be carried out at a constriction in the main gas path, wherein a respective pressure sensor is preferably arranged upstream and downstream from the constriction.

In addition, further sensors can be present, in particular one or more sensors for measuring moisture. Sufficient removal of moisture from the air can be important for the reliability of the measurement results. Sufficient removal of moisture from the exhaled air can be checked and monitored by a moisture sensor. Moreover, measured values of the moisture content of the measuring gas can be taken into account in the evaluation of the measurement data. A moisture sensor can, for example, be arranged upstream from the point where the flushing gas path branches off, so as to ensure that moisture is removed sufficiently from the flushing air. By sufficient removal of moisture from the flushing air, it is possible to avoid a situation where condensation of moisture from the flushing air in the measuring chamber negatively influences the accuracy of the nitrogen oxide measurement.

Finally, the disclosure comprises the use of the described device according to the disclosure for measuring nitrogen oxides in the exhaled air. Since the nitrogen oxides, in particular nitrogen monoxide, in the air exhaled by a person or by an animal are an important indicator of the course of asthmatic diseases and generally of inflammatory reactions in the airways or in the lungs, the course of an asthmatic disease can be measured using the device according to the disclosure. In particular, the actual reaction of the body can be monitored, so as to be able to respond accordingly, for example by administering medication. Regarding further features of the device that is used for this purpose, reference is made to the above description.

### BRIEF DESCRIPTION OF THE DRAWINGS

Further features and advantages of the disclosure will become clear from the following description of illustrative embodiments in conjunction with the drawings. The individual features can be embodied each on its own or in combination with one another.

In the drawings:

FIG. 1 shows a preferred embodiment of a device according to the disclosure for measuring nitrogen oxides in exhaled air, and

FIG. 2 shows another preferred embodiment of a device according to the disclosure for measuring nitrogen oxides in exhaled air.

### DETAILED DESCRIPTION

The device according to the disclosure is a respiratory gas analysis appliance in which a person, or an animal, in particular a patient, blows into the appliance. Some of the exhaled air is conveyed into a measuring chamber with a sensor, wherein the nitrogen monoxide content or other components in the exhaled air are determined directly or indirectly in the measuring chamber. Some of the air that is not conveyed into the measuring chamber is conveyed via a measuring gas path back out of the appliance, such that comfortable exhalation is possible for the patient. An essential aspect of the disclosure is that, on the one hand, the measuring gas path, which conveys a fraction of the exhaled air into the measuring chamber, branches off from the main gas path. Furthermore, a flushing gas path also branches off from the main gas path and in particular conveys filtered air into the measuring chamber, so as to be able there to perform flushing and, if appropriate, a zero line adjustment or a calibration of the sensor. The sampling for the measuring gas and for the flushing air takes place via at least one pump. If appropriate, two pumps can be provided, i.e. one pump in the measuring gas path and one pump in the flushing gas path. The active pumping of the respective gas fractions permits an exact control of the measurement, wherein a precisely adapted air fraction and a defined air volume can be used that are optional for the measurement. Overall, the measurement of nitrogen oxide is thereby simplified and made more precise, since exact control of the sampling in respect of the time and the quantity is possible via the pump(s). It is expedient for only a small fraction, for example 10%, of the respiratory air to be conveyed into the measuring gas path. In this way, the measuring gas path including its components (conduits, valves, pumps, measuring chamber, optionally a converter, etc.) can be made substantially smaller compared to conventional setups. The device according to the disclosure or the respiratory gas analysis appliance can thus be produced at less cost and can be designed for manual use. If a converter is provided for conversion of nitrogen monoxide, the converter can be made much smaller, and much less oxidizing agent or catalyst material is needed by comparison with what are otherwise usually passive solutions.

A particular advantage of the solution according to the disclosure is that it is possible to avoid condensation of moisture in the measuring chamber which can lead to a false measurement result on account of absorption of nitrogen dioxide in the condensed water. This is achieved by the fact that the flushing air originates from the main gas path, where dehumidifying takes place or has already taken place. The dehumidifying can preferably already take place in the mouthpiece. In previous solutions from the prior art, the respiratory air used for the measurement is dehumidified, but the flushing air, which originates from the environment, is not dehumidified.

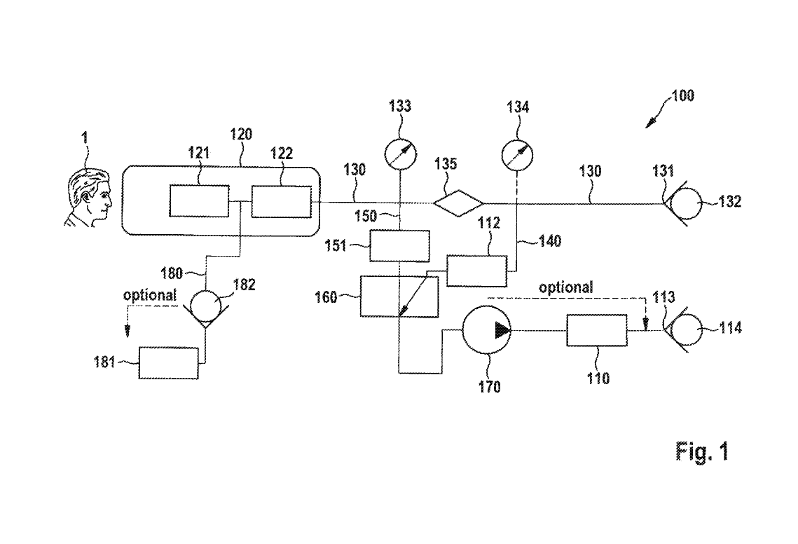

FIG. 1 is a schematic illustration of a possible embodiment of the respiratory gas analysis appliance 100 according to the disclosure. The device 100 comprises a measuring chamber 110 as gas sensor unit, which contains a gas sensor for measuring nitrogen dioxide. Furthermore, a converter 112 is assigned to the measuring chamber 110, wherein the converter 112 permits a conversion of the nitrogen monoxide from the exhaled air to nitrogen dioxide. The converter can be located at different positions in the device and in particular at different positions in the measuring gas path 140. The device 100 further comprises a mouthpiece 120, in particular an exchangeable mouthpiece or a disposable mouthpiece. The mouthpiece 120 preferably contains a microbe filter 121 and a dehumidifier 122.

A person or a patient 1 blows the exhaled air into the mouthpiece 120. The air flow is conveyed through the appliance via the main gas path 130 to an outlet opening 131, which is protected by a nonreturn valve 132 against contamination during storage. Moreover, during inhalation, the nonreturn valve 132 also prevents unfiltered air entering the appliance through the inhalation air filter 181 explained further below. A flushing gas path 150 and a measuring gas path 140 branch off from the main gas path 130. A switchover valve 160 makes it possible to switch between the measuring gas path 140 and the flushing gas path 150. A fraction of the exhaled air is branched off from the main gas path 130 via the measuring gas path 140 and the flushing gas path 150 and is conveyed into the measuring chamber 110. For the measuring or flushing, a fraction of 10%, for example, of the exhaled air can be branched off and conveyed through the measuring chamber 110. A filter 151 (zero air filter), for example an activated carbon filter, is provided in the flushing gas path 150 for the purpose of generating air substantially free of harmful substances. The flushing and, if appropriate, a zero line adjustment of the gas sensor in the measuring chamber 110 take place with the filtered air.

After it has passed through the measuring chamber 110, the measuring air or if appropriate the flushing air leaves the appliance through the outlet opening 113, which is protected by a nonreturn valve 114 against contamination during storage.

The flow of gas through the measuring gas path 140 and through the flushing gas path 150 is controlled by the pump 170. In this embodiment, the pump is arranged upstream from the measuring chamber 110. However, provision can also advantageously be made that the pump 170 is arranged downstream from the measuring chamber 110. This, on the one hand, prevents soiling of the measuring chamber 110 by contamination from the pump 170. In addition, this arrangement allows an underpressure to be built up in the measuring chamber 110, which can accelerate the flushing and the regeneration of the sensor in the measuring chamber.

For optimal control of the system, sensors 133 and, optionally, 134 are provided in the main gas path 130 and are arranged upstream and downstream from a constriction 135. The optional sensor 134 is indicated by a broken line. The sensors 133 and 134 are in particular pressure sensors via which, for example, a differential pressure measurement can be performed, such that the flow of gas or the volumetric flow rate can be controlled. The quantity of air is measured here by the pressure drop at the constriction 135 in the main gas path 130. It is also possible that one or more sensors are provided only in the position 133 or at another location.

Optionally, a further path 180 (inhalation air path) for the delivery of ambient air can open into the mouthpiece. By way of the path 180 and its inlet, the person 1 can breathe in ambient air which is purified by means of a filter 181 (zero air filter) as inhalation air filter and freed of harmful substances. A nonreturn valve 182 protects the filter 181 from contamination during storage. The nonreturn valve 182 can be arranged upstream or downstream from the filter 181. Since it is thereby possible to inhale air that is free of harmful substances, an offset of the measurement results by harmful substances from the ambient air can be ruled out.

The device or the respiratory gas analysis appliance can, for example, be designed such that a pressure drop of 3 to 5 mbar can take place during inhalation and a pressure drop of between 5 and 20 mbar can take place during exhalation.

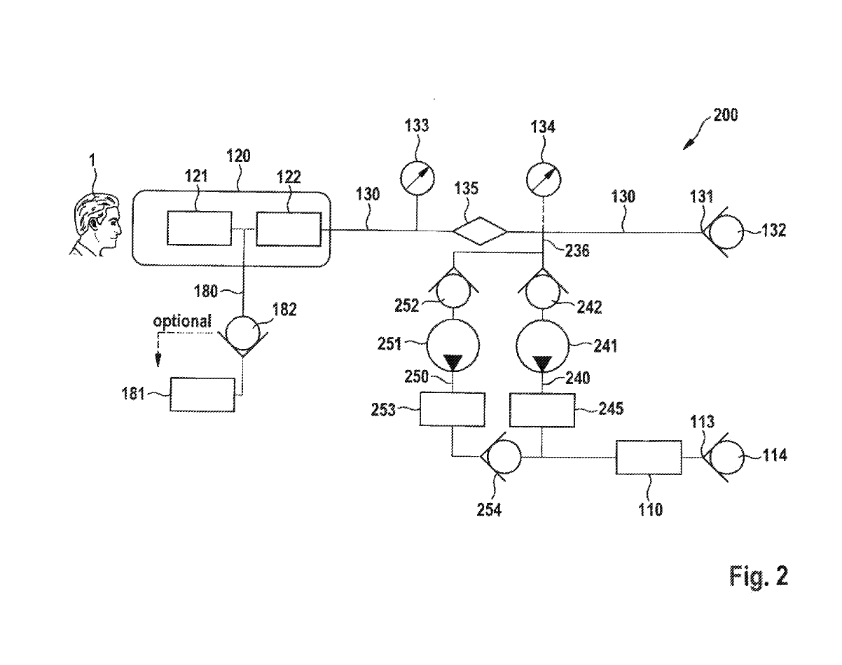

FIG. 2 illustrates a further example of an embodiment of the respiratory gas analysis appliance 200 according to the disclosure, in which a respective pump 241, 251 is provided in the measuring gas path 240 and in the flushing gas path 250. Except for the design of the measuring gas path 240 and of the flushing gas path 250, the device 200 corresponds to the device 100 illustrated in FIG. 1. The corresponding elements are therefore designated by the same reference signs as in the device 100, and reference is made in this connection to the above description. In the device 200, in contrast to the device 100, only one path 236 branches off downstream from the constriction 135, which path 236 branches into the flushing gas path 250 and the measuring gas path 240. Both in the measuring gas path 240 and also in the flushing gas path 250, a respective pump 241, 251 is provided which in each case is arranged upstream from a nonreturn valve 242, 252. A filter 253, in particular an activated carbon filter, is located downstream from the pump 251 in the flushing gas path 250 for the purpose of generating air that is substantially free of harmful substances. A further nonreturn valve 254 is located downstream from this filter. The converter 245 is located downstream from the pump 241 in the measuring gas path 240. Depending on the control of the pumps 251 and 241, a fraction of the exhaled air is conveyed from the main gas path 130 through the flushing gas path 250 or the measuring gas path 240. The flushing gas or the measuring gas then passes into the measuring chamber 110 for measurement or flushing, before it leaves the appliance via the outlet 113.

In other embodiments of the device according to the disclosure, it is also possible that, in a configuration with two pumps, i.e. a pump for the flushing gas path and a pump for the measuring gas path, one of these paths branches off upstream from a constriction in the main gas path and the other path branches off downstream from the constriction. In principle, the sequence of the branching-off paths can also be chosen freely. This also applies to embodiments with only one pump and one switchover valve. The positioning of the filter in the flushing gas path and of the various nonreturn valves can also be different and, for example, can be chosen depending on the space available in the appliance design.

What is claimed is:

1. A device for analyzing exhaled air comprising:
a gas sensor unit; and
a main gas path configured to guide air in the device and from which at least one measuring gas path and at least one flushing gas path branch off.

2. The device according to claim 1, further comprising:
at least one filter located in the at least one flushing gas path and configured to generate air that is substantially free of harmful substances.

3. The device according to claim 1, further comprising:
at least one pump configured to withdraw gas from the main gas path into the at least one measuring gas path and into the at least one flushing gas path.

4. The device according to claim 1, further comprising:
a switchover valve configured to switch between the at least one measuring gas path and the at least one flushing gas path.

5. The device according to claim 3, wherein the at least one pump is located downstream from the gas sensor unit.

6. The device according to claim 1, further comprising:
at least one first pump located in the at least one measuring gas path; and
at least one second pump located in the at least one flushing gas path.

7. The device according to claim 3, wherein the at least one pump is at least one of a diaphragm pump and a piezoceramic microblower.

8. The device according to claim 3, further comprising:
at least one nonreturn valve assigned to the at least one pump.

9. The device according to claim 1, further comprising:
a mouthpiece configured to introduce exhaled air into the device, the mouthpiece including at least one of a dehumidifier and a microbe filter.

10. The device according to claim 9, wherein a delivery path for delivery of ambient air opens into the mouthpiece, and the device further comprises:
at least one filter located in the delivery path and configured to generate air that is substantially free of harmful substances.

11. The device according to claim 10, further comprising:
at least one nonreturn valve assigned to the at least one filter.

12. The device according claim 1, further comprising:
a converter configured to at least partially convert nitrogen monoxide to nitrogen dioxide, the converter assigned to the gas sensor unit,
wherein the gas sensor unit comprises at least one gas sensor configured to measure nitrogen oxides, and
wherein the device is configured to measure nitrogen oxides in exhaled air.

13. The device according to claim 1, wherein the gas sensor unit comprises a field effect transistor.

14. The device according to claim 1, further comprising:
one or more sensors configured to measure at least one of a quantity of air, a pressure of air, a flow of air, and a humidity of air.

15. A method of using a device for analyzing exhaled air including a gas sensor unit, and a main gas path configured to guide air in the device and from which at least one measuring gas path and at least one flushing gas path branch off, the method comprising:
using the device to measure nitrogen oxides in exhaled air.
